(19) United States
(12) Patent Application Publication
(10) Pub. No.: US 2024/0000311 A1
(43) Pub. Date: Jan. 4, 2024

(54) METHOD AND ARRANGEMENT FOR DETERMINING OPTICAL ABERRATIONS AND/OR THE TOPOGRAPHY, AND/OR RECORDING REFERNECE IMAGES OF AN EYE

(71) Applicant: Carl Zeiss Meditec AG, Jena (DE)

(72) Inventors: Christian Münster, München (DE); Gregor Stobrawa, Jena (DE)

(21) Appl. No.: 18/251,065

(22) PCT Filed: Oct. 25, 2021
(86) PCT No.: PCT/EP2021/079486

§ 371 Date: Apr. 28, 2023

(30) Foreign Application Priority Data
Oct. 29, 2020 (DE) 10 2020 213 609.2

(51) Int. Cl. A61B 3/107; A61B 3/10; A61B 3/00; H04N 23/957; H04N 23/56

(52) U.S. Cl. CPC A61B 3/107; A61B 3/1015; A61B 3/0008; H04N 23/957; H04N 23/56

(57) ABSTRACT

Using the present solution, it is possible both to determine optical aberrations and/or the topography and to simultaneously or immediately successively record reference images of an eye. Within the scope of the method, the eye is illuminated with different illumination patterns by means of an illumination unit, and the light reflected by the eye is recorded by a plenoptic camera sensor and evaluated by a control and evaluation unit. According to the invention, the eye is illuminated with different illumination patterns which differ in respect of their intensity distribution and illumination direction, the light reflected by the eye is imaged onto the plenoptic camera sensor and the topography and/or optical aberrations and/or reference images of the illuminated eye are determined from the image data of the plenoptic camera sensor on the basis of the utilized illumination pattern. Even though the solution is especially provided for applications in ophthalmology, it can also be applied in other specialist fields and in industry.

### RELATED APPLICATIONS

This application claims priority from Application PCT/EP2021/079486, filed Oct. 25, 2021, and claims priority from DE Patent Application No. 10 2020 213 609.2 filed Oct. 29, 2020 each of which are incorporated by reference in their entireties in this application.

### TECHNICAL FIELD

Embodiments of the present invention relate to a method and arrangement which makes it possible both to determine imaging aberrations and/or the topography and to record reference images of an eye, where this can be done in particular simultaneously or directly successively.

### BACKGROUND

According to the prior art, there are a large number of solution variants both for determining imaging aberrations and the topography and for recording reference images of an eye.

Imaging aberrations of the human eye are captured by application of wavefront measurements (also called aberrometry). One conventional form of representation is the description of that wavefront in the corneal plane which leads to an extremely small focus on the retina. By way of example, Shack-Hartmann sensors or interferometers can be used for determining this wavefront.

In an example, the measurement of the wavefront is based on Shack-Hartmann sensors which ascertain the direction of the incident beams with a low spatial resolution of 256×256 pixels, for example.

In the interferometric measurement methods, an interferometer is used to convert the wavefront information into bright-dark information that can be detected by traditional cameras. The resolution can then be increased by way of the phase difference between measurement and reference waves, according to the traditional principle of an interferometer.

The data obtained from the wavefront measurement often form the basis of eye laser treatments used to correct defective vision.

Essentially the following three principles have become accepted for capturing and analyzing the topography of the cornea of an eye.

The Scheimpflug principle involves determining the topography by measuring the scattered light of a spatially variable projected imaging. This measurement principle very often finds application in slit lamps, since the spatially variably projected slit imaging can be used in this case.

In contrast thereto, in the case of the keratometer principle, a regular pattern at a known distance is projected onto the eye and from the reflection thereof the distortion is determined and the topography is ascertained therefrom. By way of example, concentric rings, a stripe pattern, a grid or the like are/is used as regular patterns.

The third measurement principle is based on a structured, parallel illumination which impinges on the eye from as many directions as possible and the imaging of the reflection from the eye is analyzed.

What turns out to be disadvantageous in the case of all three measurement principles is that a separate image sensor is absolutely necessary in order to be able to record and evaluate the reflections or the scattered light. If reference images of parts of the eye, such as the anterior eye portions or the fundus, for example, are additionally intended to be recorded, then a further image sensor is necessary. CCD or CMOS sensors generally find application for this purpose, which yield different color recordings or else black/white recordings.

Further disadvantages arise primarily for devices that combine measurement principles mentioned above.

By way of example, if a wavefront measurement is intended to be combined with the measurement of the topography, a wavefront sensor, e.g. Shack-Hartmann, is also required besides a CCD/CMOS sensor. In order to be able to carry out collinear measurement and simultaneous observation in this case, a beam splitter is furthermore necessary.

In this case, the beam splitters are generally also configured as color splitters in order to be able to split the illumination into different spectral ranges. While the IR range is normally used for wavefront measurements, the VIS range of light is utilized for the measurement of the topography.

The use of one or else a plurality of beam splitters and sensors increases not only the alignment complexity but also the structural space required therefor. Moreover, additional costs are caused by the sensors themselves and the evaluation units necessary in each case.

In this respect, FIG. 1 shows the basic illustration of an arrangement for determining imaging aberrations and/or the topography and/or for recording reference images of an eye according to the prior art.

For determining the topography, the cornea 1 of the eye 2 is illuminated with a pattern laterally, preferably from different directions, by the illumination unit 3 and the image of the pattern reflected from the cornea 1 is recorded by a camera sensor 5 and evaluated by a control unit 6.

In contrast thereto, for determining the wavefront, the eye 2 is illuminated axially by the illumination unit 4 in order to generate a light spot in the fovea 7. The scattered light thus generated is imaged onto the Shack-Hartmann sensor 8 and evaluated by the control unit 6. For recording reference images, the eye 2 can be illuminated both by the illumination unit 3 and by the illumination unit 4 and can be recorded by the camera sensor 5.

Plenoptic cameras, also called light field cameras, have been commercially available for some time and are also increasingly finding application in ophthalmology, inter alia.

Such cameras capture a further image dimension, namely the direction of the incident light beams, besides the usual two image dimensions. By virtue of the additional dimension, plenoptic recordings contain information about the image depth. The particular advantage of plenoptic cameras resides in the theoretically infinite depth of field and the possibility of refocusing, i.e. the subsequent displacement of the focal plane in the object space (focus variation). By virtue of the additional depth information, a plenoptic camera can also be used as a 3D camera. What is crucial for this function is that the same scene is captured from a plurality of viewing angles.

By application of an array of microlenses, or a lens grid, arranged upstream of the image sensor, each image point is refracted again and extended to form a cone that impinges on the sensor area in a circular fashion. This reveals the direction from which the light beam originally came: A perpendicularly impinging light beam lands in the center of the circle, and an obliquely incident light beam lands further at the edge. Software can thus be used to subsequently recalculate the sharpness and change the focal point as in the case of a conventional objective lens. The information from a scene has to be imaged on a plurality of pixels of the camera chip in order that the information about the direction of the incident light beam can be utilized.

The prior art discloses some solutions for ophthalmological applications which provide for using plenoptic cameras.

In this regard, US 2014/0268041 A1 describes a solution in which tomography data of eyes are determined with the aid of plenoptic imaging. For this purpose, the system has a set of light sources for illuminating the eye. The plenoptic detector is configured to record images of the light sources which are reflected from the surfaces of the eye. In particular, the intensity, position and direction of the light impinging on the detector are captured in this case. The plenoptic image data are analyzed by a processing system and the tomography data for the eye are determined.

The solution described in US 2014/0268043 A1 utilizes plenoptic imaging for the pachymetry of the cornea of eyes. Here the cornea of the eye is illuminated by a light source. The plenoptic detector is positioned at an angle relative to the eye and is configured to record an image of the light source reflected by the cornea. A processing system analyzes the plenoptic image data and determines the corneal thickness of the eye.

In US 2014/0268044 A1, a plenoptic detector is used to determine the topography of eyes. In addition, the solution is also suitable for aberrometry in order also to determine the so-called higher-order aberrations besides the standard refractive errors (myopia, astigmatism, hyperopia). For this purpose, besides a plenoptic detector and a processing unit, the system has two sets of light sources for selectively illuminating the eye. The plenoptic detector is configured to selectively receive images of the first set of light sources which are reflected from a corneal surface of the eye. The processing unit analyzes the first plenoptic image data and determines topography data of the eye. The plenoptic detector is furthermore configured to receive images of the second light source which are reflected from the retina of the eye. The processing unit analyzes the second plenoptic image data and determines aberrometry data of the eye.

US 2016/0278637 A1 describes a multimode fundus camera which yields three-dimensional and/or spectral and/or polarization-dependent recordings of the interior of the eye. The multimode fundus camera comprises a first imaging system in order to generate an optical image of an interior of an eye, and a second imaging system, which has a microlens array and a sensor array and captures a plenoptic image of the interior of the eye. Furthermore, the multimode fundus camera comprises a filter module having a plurality of filters, such as: spectral filters, polarization filters, neutral density filters, clear filters or combinations of these. As a special embodiment, a description is given of a multimode imaging system as a conversion kit enabling existing fundus cameras to be converted to a multimode fundus camera, in which the conventional sensor is replaced by a plenoptic sensor module with a microimaging array and a sensor array.

US 2017/0105615 A1 describes an imaging platform for in-vivo measurements of the eye of an individual in order to create an optical model of the eye of an individual. For this purpose, the platform comprises a plenoptic ophthalmic camera and an illumination module. According to a first configuration, the plenoptic ophthalmic camera yields an image of the corneal anterior surface of the individual's eye. In a second application, the plenoptic ophthalmic camera operates as a wavefront sensor to measure a wavefront produced by the individual's eye. The optical model of the eye is generated on the basis of the plenoptic image and the measured wavefront.

### SUMMARY OF THE INVENTION

Example embodiments of the invention include a system which makes it possible both to determine imaging aberrations and the topography of an eye and to record reference images. In this case, the system is intended to avoid the disadvantages of the solutions known according to the prior art, to have a set-up that is as simple, compact and cost-effective as possible, and to be easy to handle.

An example embodiment includes a method for determining imaging aberrations and/or the topography and/or for recording reference images of an eye, the eye being illuminated with different illumination patterns by an illumination unit, the light reflected by the eye being recorded by a plenoptic camera sensor and evaluated by a control and evaluation unit, by virtue of the fact that the eye is illuminated with different illumination patterns which differ in respect of their intensity distribution and illumination direction, that the light reflected by the eye is imaged onto the plenoptic camera sensor, and that the topography and/or imaging aberrations and/or reference images of the illuminated eye are/is determined from the image data of the plenoptic camera sensor depending on the illumination pattern used.

In this example, the arrangement for determining imaging aberrations and/or the topography and/or for recording reference images of an eye includes an illumination unit, a plenoptic camera sensor and a control and evaluation unit.

According to example embodiments of the invention, the illumination unit is configured to illuminate the eye with different illumination patterns which differ in respect of their intensity distribution and illumination direction. The plenoptic camera sensor is configured to record the light reflected by the eye.

The control and evaluation unit is configured to control the illumination unit for generating different illumination patterns which differ in respect of their intensity distribution and illumination direction. The control and evaluation unit is furthermore configured to determine the topography and/or imaging aberrations and/or to generate reference images of the illuminated eye from the image data of the plenoptic camera sensor depending on the illumination pattern used.

The present example of the invention makes it possible both to determine imaging aberrations and/or the topography and to record reference images of an object, where this can be done in particular simultaneously or directly successively. Although the solution is provided specifically for applications in ophthalmology, it can also be applied to other technical fields and industry.

### BRIEF DESCRIPTION OF THE DRAWINGS

The invention is described in more detail below on the basis of example embodiments. In this respect, in the figures:

FIG. 1: depicts a basic illustration of an arrangement for determining imaging aberrations and/or the topography and/or for recording reference images of an eye according to the prior art and

FIG. 2: depicts a basic illustration of an arrangement according to the invention for determining imaging aberrations and/or the topography and/or for recording reference images of an eye.

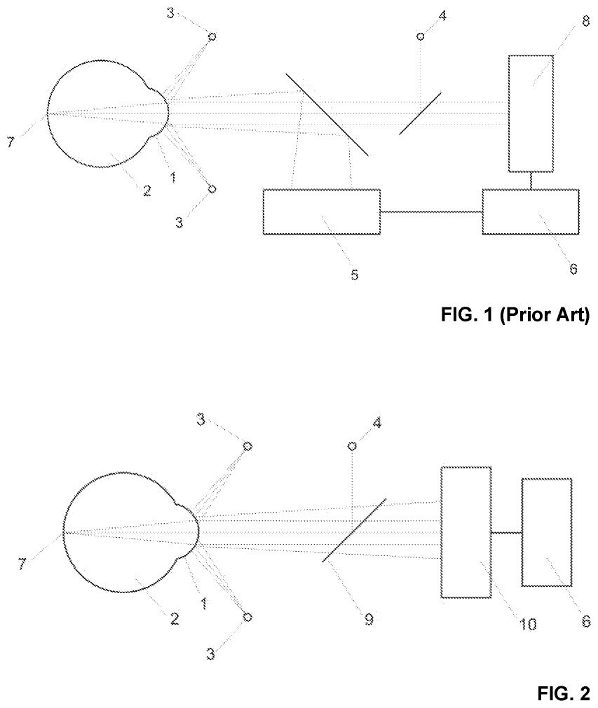

### DETAILED DESCRIPTION

In the proposed example method for determining imaging aberrations and/or the topography and/or for recording reference images of an eye, the eye is illuminated with different illumination patterns by an illumination unit, the light reflected by the eye is recorded by a plenoptic camera sensor and evaluated by a control and evaluation unit.

According to an example embodiment of the invention, the eye is illuminated with different illumination patterns which differ in respect of their intensity distribution and illumination direction, and the light reflected by the eye is imaged onto the plenoptic camera sensor. The topography and/or imaging aberrations and/or reference images of the illuminated eye are/is then determined from the image data of the plenoptic camera sensor depending on the illumination pattern used.

According to a first example configuration of the method, the eye is illuminated with an illumination pattern with a homogeneous intensity distribution from a delimited direction and imaging aberrations of the eye are determined from the image data of the plenoptic camera sensor.

According to a second example configuration of the method, the eye is illuminated with an illumination pattern with a structured intensity distribution from many directions and the topography of the eye is determined from the image data of the plenoptic camera sensor.

According to a third example configuration of the method, the eye is illuminated with an illumination pattern with a homogeneous intensity distribution from many directions and reference images of the eye are generated from the image data of the plenoptic camera sensor.

According to a further, example configuration of the method, the eye is illuminated with different illumination patterns which not only differ in respect of their intensity distribution and illumination direction but also differ in respect of their spectral characteristics.

This spectral splitting additionally makes it possible simultaneously to illuminate the eye with the different types of illumination required:
homogeneous intensity distribution from many directions for reference image,
structured intensity distribution from many directions for topography and
homogeneous intensity distribution from a delimited spatial direction for wavefront, to capture same in the plenoptic camera sensor, to extract the respectively associated information from the image data thereof, and to determine the topography and imaging aberrations and also to generate reference images of the eye.

In the simplest case, an RGB light source is used for the illumination of the eye. The spectral splitting is then effected just by splitting the three color channels R, G and B.

Example combination possibilities are discussed in greater detail below.

In accordance with a first example combination variant, the eye is illuminated with a first illumination pattern with a homogeneous intensity distribution from a delimited direction and with a second illumination pattern with a structured intensity distribution from many directions, the two illumination patterns each having different spectral characteristics. For example, the R channel is used for a wavefront analysis for determining imaging aberrations and the other two channels (G and B) are used for determining the topography. Imaging aberrations and the topography of the eye are determined from the image data of the plenoptic camera sensor depending on the respective illumination pattern and the spectral characteristic thereof.

In the case of this first example combination variant, it is possible to capture a simultaneous measurement of the corneal and ocular wavefront by subtraction of the wavefront aberrations of the interior of the eye. In combination with the measurement of the eye length, optimized IOL adaptations are possible as a result.

In accordance with a second example combination variant, the eye is illuminated with a first illumination pattern with a homogeneous intensity distribution from a delimited direction and with a second illumination pattern with a homogeneous intensity distribution from many directions, the two illumination patterns here each having different spectral characteristics. Here, too, the R channel is used for determining imaging aberrations. The other two channels (G and B) remain for generating the reference images of the eye. Imaging aberrations are determined and reference images of the eye are generated from the image data of the plenoptic camera sensor depending on the respective illumination pattern and the spectral characteristic thereof.

In accordance with a third example combination variant, the eye is illuminated with a first illumination pattern with a structured intensity distribution from many directions and with a second illumination pattern with a homogeneous intensity distribution from many directions, the two illumination patterns here, too, each having different spectral characteristics. In the case of this combination, there is no preferred variant for the splitting of the color channels. The topography is determined and reference images of the eye are generated from the image data of the plenoptic camera sensor depending on the respective illumination pattern and the spectral characteristic thereof.

In the case of the second and third example combination variants, both the focal plane for determining the topography and the wavefront for determining imaging aberrations traditionally lie on the vertex of the cornea. In contrast thereto, reference images are traditionally generated by focusing on the iris, the limbus or the sclera, which has the effect that the reference images are not sharp.

The particular advantage of plenoptic cameras is manifested here. As already described above, these cameras afford the possibility of refocusing, i.e. the subsequent displacement of the focal plane in the object space (focus variation). This is done subsequently by software.

In accordance with a fourth, example combination variant, the eye is illuminated simultaneously with three illumination patterns with a homogeneous or structured intensity distribution from a delimited direction or from many directions, the illumination patterns in this case each having different spectral characteristics. Here, too, the R channel is used for determining imaging aberrations. One of the remaining two channels (G or B) is used for determining the topography and the respective other channel (G or B) is used for generating the reference images of the eye. Imaging aberrations and the topography are determined and reference images of the eye are generated from the image data of the plenoptic camera sensor depending on the respective illumination pattern and the spectral characteristic thereof.

This fourth example combination variant is already of particular importance by virtue of the possibility of simultaneously determining imaging aberrations and the topography and generating reference images of the eye. The spatial information of the diagnosis data collected (simultaneously) in this way is essential in particular for laser-based therapy, such as topography- or wavefront-guided laser vision corrections, for example.

The arrangement for determining imaging aberrations and/or the topography and/or for recording reference images of an eye includes an illumination unit, a plenoptic camera sensor and a control and evaluation unit.

According to an example of the invention, the illumination unit is configured to illuminate the eye with different illumination patterns which differ in respect of their intensity distribution and illumination direction.

The plenoptic camera sensor is suitable for recording the light reflected by the eye. The control and evaluation unit is firstly configured to control the illumination unit for generating different illumination patterns which differ in respect of their intensity distribution and illumination direction. The control and evaluation unit is secondly configured to determine the topography and/or imaging aberrations and/or to generate reference images of the illuminated eye from the image data of the plenoptic camera sensor depending on the illumination pattern used.

In particular, the illumination unit is configured to generate illumination patterns with a homogeneous intensity distribution from a delimited direction, or with a structured intensity distribution from many directions or with a homogeneous intensity distribution from many directions.

According to one example configuration, the illumination unit is furthermore configured to simultaneously generate a plurality of illumination patterns with a different intensity distribution, illumination direction and each with a different spectral characteristic. For this purpose, the control and evaluation unit is configured to control the illumination unit accordingly. The control and evaluation unit is furthermore configured to simultaneously or directly successively determine the topography and/or imaging aberrations and/or generate reference images of the illuminated eye from the image data of the plenoptic camera sensor depending on the illumination pattern used and the spectral characteristic thereof.

According to a further advantageous configuration, the illumination unit has a polychromatic light source, the spectral components of which are splittable for the different spectral characteristics. The use of an RGB light source, the three color channels of which are separable, is particularly advantageous here for example.

In this respect, FIG. 2 shows the basic illustration of an arrangement according to the invention for determining imaging aberrations and/or the topography and/or for recording reference images of an eye.

For determining the wavefront, the eye 2 is illuminated collinearly and parallel to the optical axis of the plenoptic camera sensor 9 by the illumination unit 4 in order to generate a light spot in the fovea 7. Preferably, the non-visible, infrared part of the light spectrum is used for this purpose.

For determining the topography, the cornea 1 of the eye 2 is illuminated by the illumination unit 3 in a laterally structured manner by way of a beam splitter 9. For generating a structured illumination, the illumination unit 3 has attachment lenses (not illustrated) having a collimating effect, which are preferably arranged in a pivotable fashion.

For recording reference images, the cornea 1 of the eye 2 is likewise illuminated by the illumination unit 3, although homogeneously.

For this purpose, the illumination unit 3 has attachment lenses (not illustrated) having a non-collimating effect or has no attachment lenses.

The light reflected by the eye 2 during the respective measurements is recorded by the plenoptic camera sensor 10 and evaluated by the control unit 6. In this case, a separate evaluation of the color channels RGB enables simultaneous measurements and image recordings.

The solution according to an example embodiment of the invention provides a method and an arrangement for determining imaging aberrations and/or the topography and/or for recording reference images of an eye which enable this to be done simultaneously or directly successively.

The proposed solution makes it possible both to determine imaging aberrations and the topography of an eye and to record reference images simultaneously, the arrangement according to the invention being distinguished by a simple, compact and cost-effective set-up and easy handling.

The combination of a plenoptic camera with an illumination unit which can generate different illumination patterns which differ in respect of their intensity distribution, illumination direction and spectral characteristic makes it possible to simultaneously determine imaging aberrations and topography and also to generate reference images of the eye. According to the invention, moreover, only one image sensor is used for this purpose.

These simultaneously realized recordings additionally have the advantage the possible eye movements need not be considered and the assignment of wavefront, topography and reference image is thus significantly simplified.

Since all the recordings are realized by way of the same reference system, there is an intrinsically identical scaling, no offset, no rotation or no further alignment errors of the images.

1.-13. (canceled)

14. A method for determining at least one of aberrations of an eye, topography of an eye, for recording reference images of an eye or a combination of the foregoing, comprising
illuminating the eye with a lighting unit with different lighting patterns;
recording light reflected from the eye with a plenoptic camera sensor;
evaluating the light recorded by the plenoptic camera sensor by application of a control and evaluation unit;
illuminating the eye with different illumination patterns that differ in terms of intensity distribution and direction of illumination;
imaging the light reflected by the eye of the different illumination patterns on the plenoptic camera sensor; and
determining from image data of the plenoptic camera sensor and the different illumination patterns, at least one of the topography, the aberrations and the reference images of the illuminated eye.

15. The method according to claim 14, further comprising illuminating the eye with an illumination pattern with a homogeneous intensity distribution from a delimited direction and determining imaging errors of the eye from the image data of the plenoptic camera sensor.

16. The method according to claim 14, further comprising illuminating the eye with an illumination pattern with a structured intensity distribution from many directions and determining the topography of the eye from the image data of the plenoptic camera sensor.

17. The method according to claim 14, further comprising illuminating the eye with an illumination pattern with homogeneous intensity distribution from many directions and generating reference images of the eye from the image data of the plenoptic camera sensor.

18. The method according to claim 14, further comprising illuminating the eye with two illumination patterns including a first illumination pattern with homogeneous intensity distribution from a limited direction and a second illumination pattern with structured intensity distribution from many directions, wherein the two illumination patterns each have different spectral characteristics and determining imaging errors and the topography of the eye from the image data of the plenoptic camera sensor depending on the respective illumination pattern and spectral characteristics.

19. The method according to claim 14, further comprising illuminating the eye with two illumination patterns including with a first illumination pattern with homogeneous intensity distribution from a delimited direction and a second illumination pattern with homogeneous intensity distribution from many directions, wherein the two illumination patterns each have different spectral characteristics and wherein imaging errors are determined from the image data of the plenoptic camera sensor depending on the respective illumination pattern and its spectral characteristics, and generating the reference images of the eye.

20. The method according to claim 14, further comprising illuminating the eye with two illumination patterns including a first illumination pattern with structured intensity distribution from many directions and a second illumination pattern with homogeneous intensity distribution from many directions,
wherein the two illumination patterns each have different spectral characteristics; and determining from the image data of the plenoptic camera sensor depending on the respective illumination pattern and the spectral characteristics, the topography and generating the reference images of the eye.

21. The method according to claim 14, further comprising illuminating the eye with three illumination patterns with homogeneous or structured intensity distribution from a limited direction or from many directions at the same time, wherein the illumination patterns each have different spectral characteristics and determining aberrations and the topography and generating the reference images of the eye from the image data of the plenoptic camera sensor depending on the respective lighting pattern and the spectral characteristics.

22. An arrangement that determines aberrations, that determines topography, that records reference images of an eye or a combination of the foregoing, comprising:
an illumination unit;
a plenoptic camera sensor; and
a control and evaluation unit;
wherein the illumination unit is configured to illuminate the eye with different patterns, that differ in terms of intensity distribution and direction of illumination;
wherein light reflected by the eye is imaged onto the plenoptic camera sensor;
wherein the control and evaluation unit is configured to control the illumination unit to generate different illumination patterns that differ in terms of intensity distribution and direction of illumination; and
wherein the control and evaluation unit is also configured to determine topography, imaging errors or both from the image data of the plenoptic camera sensor depending on the illumination pattern used, to generate reference images of the illuminated eye or a combination of the foregoing.

23. The arrangement according to claim 22, wherein the lighting unit is configured to generate lighting patterns with a homogeneous intensity distribution from a limited direction, or with a structured intensity distribution from many directions, or with a homogeneous intensity distribution from many directions.

24. The arrangement according to claim 22,
wherein the lighting unit is also configured to simultaneously generate a plurality of lighting patterns with different intensity distributions, lighting directions and different spectral characteristics in each case;
wherein the control and evaluation unit is configured to control the lighting unit to simultaneously generate different illumination patterns, with different intensity distribution, direction of illumination and each with different spectral characteristics; and
wherein the control and evaluation unit is also configured to simultaneously or directly successively determine the topography, determine the imaging aberrations, generate the reference images of the illuminated eye or a combination of the foregoing from the image data of the plenoptic camera sensor depending on the illumination pattern used and the spectral characteristic.

25. The arrangement according to claim 22, wherein the lighting unit has a polychrome light source having spectral components that can be divided for the different spectral characteristics.

26. The arrangement according to claim 25, wherein the polychrome light source is an RGB light source having three color channels that can be separated.
